(19) United States
(12) Patent Application Publication
(10) Pub. No.: US 2026/0174680 A1
(43) Pub. Date: Jun. 25, 2026

(54) MAMMARY GLAND TISSUE HEALTH MAINTENANCE AGENT HAVING GABA AS ACTIVE INGREDIENT

(71) Applicant: SANWA SHURUI CO., LTD., Usa-shi, Oita (JP)

(72) Inventors: Akihiro Nakamura, Usa-shi (JP); Hideki Hokazono, Usa-shi (JP); Junji lto, Osaka-shi (JP); Sunao Tanaka, Los Angeles, CA (US); Masakazu Toi, Tokyo (JP)

(73) Assignee: SANWA SHURUI CO., LTD., Usa-shi, Oita (JP)

(21) Appl. No.: 19/129,983

(22) PCT Filed: Nov. 16, 2023
(86) PCT No.: PCT/JP2023/041237

§ 371 Date: May 14, 2025

(30) Foreign Application Priority Data
Nov. 16, 2022 (JP) 2022-183497

(51) Int. Cl. A61K 9/00; A61K 31/519; A61K 31/70

(52) U.S. Cl. CPC A61K 9/0019; A61K 31/519; A61K 31/70

(57) ABSTRACT

Problem: To provide an oral agent capable of maintaining the health of mammary gland tissue.

Solution: An agent for maintaining health of mammary gland tissue is characterized by containing GABA as an active ingredient. In addition, the combined use of GABA and equol can enhance the effect of maintaining the health of mammary gland tissue and the effect of preventing early stage breast cancer by inhibiting dysplasia in mammary gland tissue.

### TECHNICAL FIELD

The present invention relates to a technical field of maintaining health of mammary gland tissue and preventing breast cancer. More specifically, the present invention relates to a health maintenance agent of a mammary gland tissue or a composition for maintaining health.

### BACKGROUND ART

The number of breast cancer patients is increasing worldwide, and breast cancer is a familiar and serious disease, affecting one in eight women in the United States and one in nine women in Japan. A major problem with breast cancer is the progression to metastatic breast cancer, which spreads to other organs, especially the liver, brain, bones, lungs, lymph nodes, skin, soft tissues, and the like. Breast cancer is a heterogeneous disease maintained by a plurality of complex proliferation pathways that are interrelated.

Breast cancer begins with the development of a proliferative benign tumor within the mammary gland, and as the tumor continues to proliferate within the mammary gland, the tumor acquires the ability to invade the stromal side of the mammary gland and becomes malignant (invasive cancer). The invading cancer cells travel through the blood and lymphatic system to metastasize to other organs and lymph nodes, forming metastatic lesions and causing death.

Although the cause of breast cancer has not yet been clarified, several factors that increase the risk of developing breast cancer are known. The main factors include excessive exposure to estrogen, which is a female hormone, age, genetic abnormalities, and the like, and there are factors related to female hormones, factors related to an individual's heredity and constitution, and factors related to lifestyle habits such as body shape, alcohol, and smoking history.

It is epidemiologically known that one of the factors involved in the development of breast cancer is estrogen, which is a female hormone, and it has also been experimentally proven that estrogen promotes the development of breast cancer. However, there are many unknowns about the early signs of tumor development and the mechanisms of early development, and the early signs of tumor development and the mechanisms of early development have not been experimentally clarified. The present inventors developed a mouse model system that develops early stage breast cancer, and used mice with reduced DNA repair ability to intraperitoneally administer estradiol (E2) to these mice for 30 days, which induced mammary gland dysplasia, thereby scientifically proving the effect of estrogen in the development of atypical proliferative mammary lesions, which are considered to be one of the early signs of breast cancer (Non-Patent Literature 1). In early stage breast cancer, proliferative atypical tumor lesions are formed, and the lesions acquire sustained proliferation capabilities and further acquire invasive capabilities. Furthermore, it was confirmed that when equol, which is an estrogen analog, was administered intraperitoneally to a mouse model system that develops early stage breast cancer, mammary gland tissue dysplasia was inhibited even when estradiol was administered, and the development of breast cancer was suppressed (Non-Patent Literature 1).

Equol is an isoflavone compound, and is an active metabolite of soy isoflavones that is produced when isoflavonoids present in soy foods are converted by intestinal microorganisms when soy foods are ingested. It has been reported that equol, which is a metabolite of soy isoflavones, is more effective than soy isoflavones in treating breast cancer, prostate cancer, anti-aging, menopausal symptoms, and postmenopausal osteoporosis (Patent Literature 1 and Non-Patent Literatures 2 to 4).

In addition, it has been reported that when a mouse model that develops early stage breast cancer is given fermented barley extract while estradiol is administered intraperitoneally to the mouse, benign proliferative tumors develop in the mammary glands, but the invasion of breast cancer is suppressed and the cancer does not become invasive, unlike when equol is administered (Non-Patent Literature 5). At the same time, since it has been confirmed that oral ingestion of fermented barley extract maintains the elastic fibers in the mammary glands, it is considered that fermented barley extract prevents the invasion of breast cancer by the action of maintaining the elastic fibers in the mammary glands.

On the other hand, y-amino butyric acid (GABA) is a non-proteinogenic amino acid that is widely distributed in nature, and is an inhibitory neurotransmitter that is abundant in the central nervous system of mammals. With the revision of the food and drug classification in 2001, it became possible to use GABA as food, and GABA is also commercially available as a supplement in the form of tablets or capsules alone. It has been reported for GABA to have a wide range of physiological activities, such as a sedative action and a blood pressure lowering action (Non-Patent Literature 6), and an action of improving skin elasticity (Patent Literature 2). Fermented barley extract contains very little GABA.

### CITATION LIST

### Patent Literatures

Patent Literature 1: PCT Japanese Translation Patent Publication No. 2015-500802
Patent Literature 2: Japanese Patent Application Laid-Open No. 2018-30826

### Non-Patent Literatures

Non-Patent Literature 1: iScience 23, 100821 Feb. 21, 2020
Non-Patent Literature 2: Biol. Reprod. (2004) Vol. 70, p. 1188-95
Non-Patent Literature 3: Jpn. J. Clin. Oncol. (2002) Vol. 32, No. 8, p. 296-300
Non-Patent Literature 4: J. Nutr. (2002) Vol. 32, No. 3, p. 595S
Non-Patent Literature 5: Acta Histochem. Cytochem. (2021) Vol. 54, No. 2, p. 73-78
Non-Patent Literature 6: Journal of the Japanese Society of Taste Technology (2010) No. 15, p. 32-37

### SUMMARY

### Technical Problem

Inside the breast, there are organs called lobules that secrete breast milk, the nipples which are the exit points for breast milk, and milk ducts that connect the lobules to the nipples. These lobules, milk ducts, and nipples are collectively called the mammary gland. The mammary gland is composed of mammary epithelial cells on the lumen side and myoepithelial cells on the stromal side. When mammary epithelial cells in the milk ducts or lobules in the breast begin to proliferate uncontrollably and invade the stromal side, the condition is called malignant breast cancer.

Invasion of breast cancer is a phenomenon in which mammary epithelial cells break through the myoepithelial cell layer and basement membrane on the stromal side and advance into the stroma. Invaded breast cancer cell group spreads within the tissue and simultaneously metastasizes to other organs via blood vessels and lymphatic vessels.

Breast cancer that proliferates only within the milk ducts is called non-invasive breast cancer, and breast cancer that has spread outside the milk ducts is called invasive breast cancer. When invasive breast cancer occurs, there is a likelihood that the cancer may metastasize to the lymphatic vessels or lymph nodes, or that cancer cells may enter the blood vessel and cause distant metastasis to the liver, lungs, bones, and the like. It is said that proliferation at the metastatic sites leads to organ dysfunction and death, and therefore, if metastasis can at least be inhibited, the risk of death will be greatly reduced.

Dysplasia is a pathological term used when determining by observing cells and tissues under a microscope, and refers to a state in which the shape of cells is different from normal, and refers to “a state that cannot currently be called cancer, but has a high likelihood of progressing to cancer (precancerous lesion)” or “a state that is on the borderline between malignant and benign (borderline malignant).”

An object of the present invention is to provide an active ingredient capable of maintaining the health of mammary gland tissue. Maintaining the health of mammary gland tissue means inhibiting the state of mammary gland tissue so that the state does not develop dysplasia, or inhibiting the progression of dysplasia. The result leads to breast cancer prevention in healthy subjects or non-healthy subjects.

Furthermore, an object of the present invention is to produce food and drink that has the effect of maintaining the health of mammary gland tissue, and to provide a supplement, a pharmaceutical product, food and drink, and feed for maintaining the health of mammary gland tissue.

### Solution to Problem

The present inventors have found that GABA inhibits dysplasia of mammary gland tissue and prevents early stage breast cancer, and based on this finding, have completed the present invention relating to the use of GABA as food and drink, a supplement, and a pharmaceutical product.

The present invention relates to a health maintenance agent for a mammary gland tissue according to the following (1) to (7) and a food and drink composition according to (8).
(1) A health maintenance agent of a mammary gland tissue, containing GABA as an active ingredient.
(2) The health maintenance agent according to the above (1), further containing equol.
(3) The health maintenance agent according to the above (1) or (2), wherein the health maintenance agent is for preventing breast cancer.
(4) The health maintenance agent according to the above (3), wherein the health maintenance agent has an action of inhibiting mammary gland tissue dysplasia and invasion of breast cancer.
(5) The health maintenance agent according to the above (1) or (2), wherein the health maintenance agent further has an action of maintaining skin elasticity.
(6) The health maintenance agent according to the above (1) or (2), wherein the health maintenance agent is a preparation, a drink, a food composition, or a pharmaceutical product to be administered orally.
(7) The health maintenance agent according to the above (1) or (2), wherein a daily ingestion amount of GABA is 10 mg to 3,000 mg.
(8) A food and drink composition for maintaining health of mammary gland tissue, containing the agent according to the above (1) or (2).

The present invention also relates to a method for maintaining health of mammary gland tissue according to the following (9), a method for inhibiting mammary gland tissue dysplasia according to the following (10), and a method for preventing breast cancer according to the following (11).
(9) A method for maintaining health of mammary gland tissue, including a step of administering GABA to a human.
(10) A method for inhibiting mammary gland tissue dysplasia and invasion of breast cancer, including a step of administering GABA to a human.

(11) A method for preventing breast cancer, including a step of administering GABA to a human.

### Advantageous Effects of Invention

According to the present invention, it is possible to provide an agent for maintaining the health of mammary gland tissue by containing GABA as an active ingredient and inhibiting dysplasia of mammary gland tissue to prevent early stage breast cancer. Furthermore, this effect can be enhanced by using GABA in combination with equol.

Additionally, it is possible to provide a supplement, a pharmaceutical product, food and drink, and feed that can be easily ingested to maintain the health of mammary gland tissue.

### BRIEF DESCRIPTION OF DRAWINGS

FIG. 1 shows results of Example 1.

Ten-week-old Scid mice were intraperitoneally administered with 6 μg of E2 per day for 25 days, and were simultaneously intraperitoneally administered with 6 μg of Equol per day and/or were intraperitoneally administered with 0.5 mg (low) or 5 mg (high) of GABA per day. The fourth mammary gland was sampled 24 hours after the final administration, and photographs are shown of tissue observation by hematoxylin and eosin (H&E) staining (upper row) and Elastica van Gieson (EVG) staining (lower row).

FIG. 2 shows the same results of Example 1. FIG. 2 shows further evaluation of the frequency of occurrence of dysplasia in sections of mammary gland tissue after staining.

FIG. 3 shows results of Example 2.

After MCF 7 (ATCC HTB-22) cells were cultured, and the medium was removed, 200 μL of PBS adjusted to E2-free, 10 nM of E2, or 10 nM of E2+0.3 μg/mL to 1.1 μg/mL of GABA was added to each well, and the cells were incubated at 37° C. and 5% of CO₂ for 4 hours. The cells were visualized by adding a primary histone antibody, and the DNA double-strand break signals per cell were measured by observation under a microscope.

### DESCRIPTION OF EMBODIMENTS

GABA, which is ingested exogenously in the present invention to maintain the health of mammary gland tissue, is an abbreviation for γ-amino butyric acid. The abbreviation GABA obtained by taking the initials of the English name, is commonly and widely used. GABA is a type of amino acid that is widely distributed in plants and animals, and the like, and is an inhibitory neurotransmitter present in the brains and spinal cord of mammals. GABA improves blood flow to the brain, increases oxygen supply, and enhances cerebral metabolism. GABA is found primarily in the brain of living bodies and is involved as a neurotransmitter in the central nervous system. GABA is known as the main inhibitory neurotransmitter and is used for the purpose of stimulating blood flow in the diencephalon, enhancing the metabolic function of brain cells, and relieving autonomic nervous system caused by stress. It is also known that GABA has an action of improving skin elasticity.

The GABA in the present invention is GABA extracted from vegetables, fruits, grains, and the like, GABA produced from fermented foods, and GABA produced by organic synthesis.

The above-mentioned vegetables, fruits, and grains refer to pumpkin, eggplant, tomato, cucumber, rice, brown rice, malt, soybeans, and the like, and fermented foods refer to fermented foods such as kimchi, pickles, fermented milk, and natto, which are derived from lactic acid bacteria, yeast, and natto bacteria. GABA can be obtained by fermenting germ rice, green tea, or rice bran with lactic acid bacteria, or by fermenting glutamic acid using lactic acid bacteria. GABA may also be obtained by enzymatic conversion of glutamic acid and/or sodium glutamate as raw materials using naturally occurring glutamic acid decarboxylase (GAD), or by isolating bacteria from fermented foods and preparing the bacteria in a culture solution.

These fermented solutions and extracted solutions may be dried and powdered by freeze-drying or spray-drying as appropriate. Although not particularly limited, the GABA content is 0.1% to 30% in liquid form and 0.1% to 99% in powder form. The daily ingestion amount of GABA for humans is 1 mg to 3,000 mg, preferably 10 mg to 3,000 mg. More preferably, the amount is from 20 mg to 1,000 mg, and even more preferably, the amount is from 100 mg to 1,000 mg. When the amount is less than 10 mg as GABA, no effect can be expected, and when the amount is equal to or more than 3,000 mg, it becomes difficult to ingest the GABA at once.

Furthermore, equol used in the present invention is an isoflavone compound, and is an active metabolite of soy isoflavones that is produced when isoflavonoids present in soy foods are converted by intestinal microorganisms when soy foods are ingested. Commercially available equol can be used.

Equol is produced by intestinal bacteria, and it is considered that the degree of production of equol varies from person to person. It is estimated that people who are unable to produce equol do not have equol-producing bacteria in their intestines, and it is considered that in such cases, even though the people ingest soy processed foods, the desired anti-estrogenic effects or estrogen-like effects cannot be expected.

In the present invention, maintaining the health of mammary gland tissue refers to inhibiting the state of mammary gland tissue so that the state does not develop dysplasia, or inhibiting the progression of dysplasia. Inside the breast, there are mammary glands consisting of lobules, milk ducts, and nipples. Breast cancer is a cancer that develops in the tissue of the mammary glands. Most cancers develop from the milk ducts, but some cancers develop from the lobules.

Dysplasia is a pathological term used when determining by observing cells and tissues under a microscope, and refers to a state in which the shape of cells is different from normal, and refers to “a state that cannot currently be called cancer, but has a high likelihood of progressing to cancer (precancerous lesion)” or “a state that is on the borderline between malignant and benign (borderline malignant).”

Dysplasia of mammary gland tissue refers to precancerous lesion or borderline malignant in the mammary gland, and while dysplasia is not necessarily fatal, dysplasia means that the mammary gland tissue is not in a healthy state. Since cells progress from dysplasia to invasive cancer cells with the passage of time, when dysplasia can be inhibited by the present invention, early stage breast cancer can be prevented; and when breast cancer can be prevented in the early stages, the development of invasive breast cancer can be avoided.

Invasion of breast cancer is a phenomenon in which mammary epithelial cells break through the myoepithelial cell layer and basement membrane on the stromal side and advance into the stroma. Invaded breast cancer cell group spreads within the tissue and simultaneously metastasizes to other organs via blood vessels and lymphatic vessels.

The inhibitory action of the present invention on dysplasia of mammary gland tissues is specifically confirmed in animal experiments using a mouse model system that develops early stage breast cancer. This mouse model system that develops early stage breast cancer is an experimental system developed by the present inventors, and a system in which scid mice, which are a mouse strain with reduced DNA repair ability, are used to be administered estradiol (E2) intraperitoneally for 30 days to induce mammary gland tissue dysplasia. Non-Patent Literature 1 describes how intraperitoneal administration of equol using this system was able to inhibit mammary gland tissue dysplasia, and the system is an experimental system that can be used to evaluate substances that have a preventive effect against breast cancer. The present invention also uses this experimental system to evaluate the effects of GABA or the combined use of GABA and equol.

The health maintenance agent for a mammary gland tissue containing GABA of the present invention, or the health maintenance agent for a mammary gland tissue containing GABA and equol, may be in the form of food and drink rich in GABA, or GABA and equol. The form of the food and drink is not particularly limited, but may be formed into a powder, granules, capsule, or tablet. In addition, other forms of the food and drink may be a food material or a food additive, or may be a form such as a syrup, a suspension, a drink, a liquid diet, a soft drink, a milk drink, a lactic acid bacteria drink, a functional seasoning, a gel food, a pudding, a yogurt, confectionery and cakes, bread, noodles, pasta, chocolate, candy, chewing gum, and the like.

Furthermore, the food and drink is not limited to food and drink for humans, but also includes feed for mammals such as dogs and cats kept as pets or livestock. The concept of food and drink includes not only ordinary foods and drinks, but also drinks, so-called supplements, health foods, enteral nutritional foods, foods for special dietary uses, foods with nutritional functions, foods for specified health uses, and the like.

One embodiment of the health maintenance agent for a mammary gland tissue of the present invention is preferably used for at least one of the following purposes: as a pharmaceutical product, food (supplement), and a food additive. When the agent is used as a pharmaceutical product, examples include, but are not particularly limited to, oral administration agents such as powders, granules, capsules, pills, and tablets.

The effective dosage of the pharmaceutical composition varies depending on the state of the subject to be administered (including age, physical state, and the like), dosage form, and the like, but the oral dosage for a human (adult weighing 60 kg) can be set within the above-mentioned range of daily ingestion amount for a human.

Next, specific examples of the present invention will be described with reference to the following examples, but the present invention is not limited to these examples.

### Example 1

### <Animal Experiment>

### [Experimental Method]

Ten-week-old scid mice were intraperitoneally administered PBS as a negative control group, 6 μg/day of E2 as a positive control group, 6 μg of E2+6 μg/day of equol (Eq) in experimental group 1, 6 μg of E2+0.5 mg/day of GABA in experimental group 2, 6 μg of E2+5 mg/day of GABA in experimental group 3, 6 μg of E 2+6 μg of Eq+0.5 mg of GABA in experimental group 4, and 6 μg of E 2+6 μg of Eq+5 mg of GABA in experimental group 5, with six mice in each group, every morning for 25 days. 24 hours after the final administration, the fourth mammary gland was sampled, and the tissue was observed by hematoxylin and eosin (H&E) staining and Elastica van Gieson (EVG) staining.

In addition, the frequency of occurrence of dysplasia in sections of the mammary gland tissue was evaluated.

### [Experimental Materials]

Mouse (female): C. B-17/Icr-scid/scid Jel was purchased from CLEA Japan.

The E2 used was E2758 manufactured by SIGMA, the Equol (Eq) used was SML2147 manufactured by SIGMA, and the GABA used was A5835 manufactured by SIGMA.

### [Results]

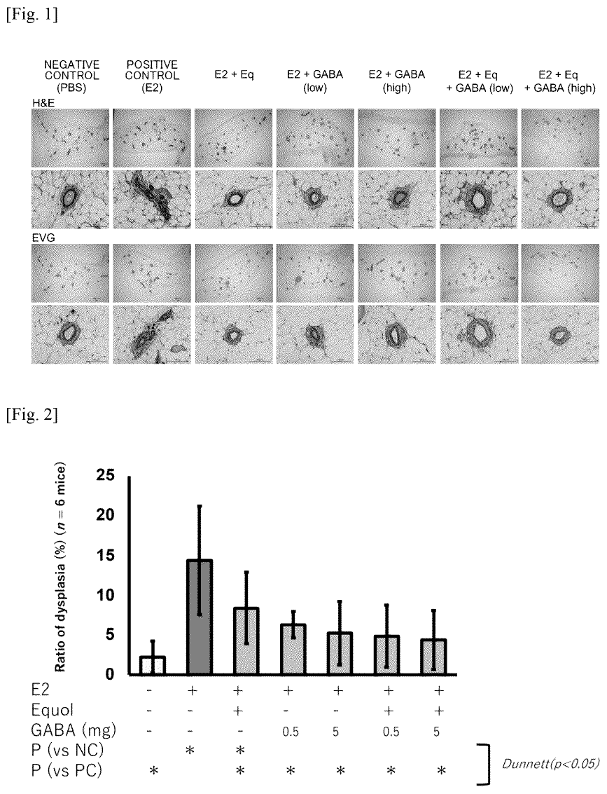

Photographs of H&E stained (upper row) and EVG stained (lower row) mammary gland tissue sections are shown in FIG. 1. The negative control containing only phosphate-buffered saline (PBS) had a normal mammary gland shape, whereas the positive control containing only E2 showed invasion. In the experimental groups 1 to 5 in which Eq or GABA was added to E2, no invasion was observed.

The frequency of occurrence of dysplasia is shown in FIG. 2. The vertical axis represents the frequency of occurrence of dysplasia.

When the average value of the positive control of E2 only was taken as 100%, the value for Eq alone (experimental group 1) was approximately 58%, the values for GABA alone (experimental groups 2 and 3) were 44% and 37%, respectively, and the values for Eq+GABA (experimental groups 4 and 5) were 34% and 30.5%, respectively. In the negative control, the value was 16%.

Furthermore, the significant differences of these values were tested by Dunnett's method. As a result, compared to the negative control, there was a significant difference (p<0.05) in Eq alone (experimental group 1), but there was no significant difference in experimental groups 2 to 5. There were also significant differences in all experimental groups compared to the positive control.

The results confirmed that in a mouse model experiment of spontaneous breast cancer, intraperitoneal administration of GABA inhibited the dysplasia and invasion of mammary gland tissue induced by estradiol (E2).

### [Consideration]

GABA alone and Eq+GABA have a higher inhibitory effect on dysplasia of mammary gland tissue than equol (Eq), which is known to have a preventive effect on breast cancer, and are therefore considered to have a preventive effect on early stage breast cancer. Among these, it was suggested that Eq+GABA may have the greatest effect.

### Example 2

### <Cell Test>

Since it is also conceivable that GABA may act directly on cells to reduce the toxicity of E2, cultured cells were used and DNA damage caused by the addition of E2 or E2 and GABA was compared using immunohistochemistry.

The cultured cells used were MCF7 (ATCC HTB-22). MCF7 cells were cultured for 2 days to 3 days in phenol red-free RPMI 1640 medium containing 10% of fetal bovine serum and 1 nM of E2.

The cells were passaged into phenol red-free RPMI medium containing 10% of charcoal-stripped serum (Charcoal-Stripped FBS, Thermo Fisher) and 1 nM of E 2, and seeded into an 8-well plate (SCS-N28 MAS-Coat, Matsunami) at 400 μL/well. After 1 day to 2 days, the medium on the 8-well plate was removed and replaced with serum-free, phenol red-free RPMI medium, and the cells were starved by culturing for 1 day to align the cell cycle to the G1 phase. The culture was carried out at 37° C. and 5% of CO₂.

After the medium was removed, 200 μL of PBS adjusted to E2-free, 10 nM of E2, 10 nM of E 2 +0.3 μg/mL of GABA, 10 nM of E2+0.6 μg/mL of GABA, or 10 nM of E2+1.1 μg/mL of GABA was added to each well, and the cells were incubated at 37° C. and 5% of CO₂ for 4 hours. 200 μL of 2% formaldehyde was added to each well so that the final concentration of formaldehyde was 1%, and the mixture was left for 15 minutes for fixation. The wells were washed three times with PBS containing 0.05% of Tween 20, and 200 μL of PBS containing 0.1% of Triton-X was added to each well and left for 15 minutes for permeabilization.

The treatment solution was removed and the wells were washed twice with PBS containing 0.05% of Tween 20. P-Histone H2AX (Cell Signaling Technology), which is the primary antibody, was then diluted 200-fold with PBS containing 5% of Blocking One Histo (Nacalai) and 0.1% of Triton-X, and 200μL of the solution was added to each well and left at 4° C. overnight.

The primary antibody solution was removed, and the wells were washed twice with PBS containing 0.05% of Tween 20. Alexa Fluor 546 (Life Technologies) was diluted 1000-fold in PBS containing 5% of Blocking One Histo and 0.1% of Triton-X, and 200 μL of the solution was added to each well and left at room temperature for 1 hour.

After washing the wells twice with PBS containing 0.05% of Tween 20, one drop of Prolong Gold (Cell signaling Technology) was added to each well and the wells were sealed with a cover glass.

The cells were observed under a microscope, the DNA double-strand break signals per cell were measured, and statistical analysis was performed using the Mann-Whitney U-test, with p<0.05 being considered to indicate a significant difference. Bonferroni correction was performed on p values.

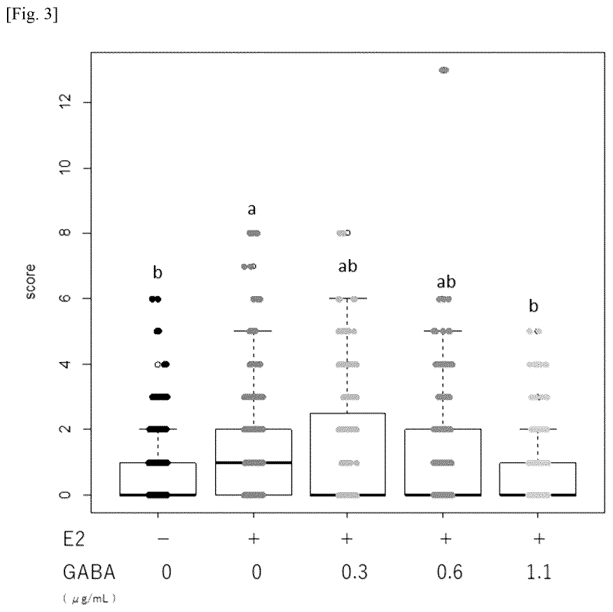

For each experiment section, DNA double-strand break signals were counted in 100 cells. The results are shown in FIG. 3.

The addition of E2 increased the number of DNA damages in the cells, whereas the addition of GABA tended to reduce the number of DNA damages even in the addition of E2, and the addition of 1.1 μg/mL of GABA significantly reduced DNA damage.

These results indicate that GABA has the effect of inhibiting E2-induced DNA damage by acting directly on cells, which is considered to be one of the mechanisms of action by which GABA inhibits dysplasia in mammary gland tissue.

1.-8. (canceled)

9. A method of maintaining health of a mammary gland tissue in a human, comprising administrating GABA to the human.

10. A method of inhibiting mammary gland tissue dysplasia in a human, comprising administrating GABA to the human.

11. A method of preventing breast cancer in a human, comprising administrating GABA to the human.

12. A method of inhibiting invasion of breast cancer in a human, comprising administrating GABA to the human.

13. The method according to claim 9, which further comprises administrating equol to the human.

14. The method according to claim 10, which further comprises administrating equol to the human.

15. The method according to claim 11, which further comprises administrating equol to the human.

16. The method according to claim 12, which further comprises administrating equol to the human.

17. The method according to claim 9, wherein the administration of GABA comprises oral administration of a GABA-containing preparation, a GABA-containing drink, a GABA-containing food composition, or a GABA-containing pharmaceutical product.

18. The method according to claim 10, wherein the administration of GABA comprises oral administration of a GABA-containing preparation, a GABA-containing drink, a GABA-containing food composition, or a GABA-containing pharmaceutical product.

19. The method according to claim 11, wherein the administration of GABA comprises oral administration of a GABA-containing preparation, a GABA-containing drink, a GABA-containing food composition, or a GABA-containing pharmaceutical product.

20. The method according to claim 12, wherein the administration of GABA comprises oral administration of a GABA-containing preparation, a GABA-containing drink, a GABA-containing food composition, or a GABA-containing pharmaceutical product.

21. The method according to claim 9, wherein a daily dosage of GABA is 10 mg to 3,000 mg.

22. The method according to claim 10, wherein a daily dosage of GABA is 10 mg to 3,000 mg.

23. The method according to claim 11, wherein a daily dosage of GABA is 10 mg to 3,000 mg.

24. The method according to claim 12, wherein a daily dosage of GABA is 10 mg to 3,000 mg.
